(19) United States
(12) Patent Application Publication
(10) Pub. No.: US 2008/0197086 A1
(43) Pub. Date: Aug. 21, 2008

(54) Assembly for the Treatment of a Polymerizable Material

(76) Inventor: Jurgen Mosler, Castrop-Rauxel (DE)

Correspondence Address: SMITH MOORE LLP, P.O. BOX 21927, GREENSBORO, NC, 27420, US

(21) Appl. No.: 11/917,050

(22) PCT Filed: Jun. 30, 2006
(86) PCT No.: PCT/EP2006/006352

§ 371 Date: May 2, 2008

(30) Foreign Application Priority Data
Apr. 11, 2005 (FI) 20050365

(51) Int. Cl. C07C 51/25; C07C 51/42; C07C 57/04; B01D 3/00

(52) U.S. Cl. 210/758; 261/155; 261/152; 261/156; 422/201; 562/600

(57) ABSTRACT

The present invention relates to an arrangement (1) for treatment of a polymerizable material (12) comprising at least one gassing device (2) to feed a gas (11) into the polymerizable material (12) and a heating device (3) to heat the polymerizable material (12) provided with gas (11), characterized in that the heating device (3) is designed such that the polymerizable material (12) flows through the heating device (3) via an inlet (4) against gravity (9) and means for even distribution of the gas (11) over the inlet (4) are provided. The invention also relates to a process for heating of a polymerizable material, a device for production of (meth)acrylic acid and a process for production of high purity (meth)acrylic acid.

This application is a national stage application under 35 U.S.C. 371 of international application No. PCT/EP2006/006352 filed 30 Jun. 2006, and claims priority to German Application No. DE 10 2005 030 416.8 filed 30 Jun. 2005, the disclosures of which are expressly incorporated herein by reference.

### BACKGROUND

The present invention relates to an arrangement for treatment of a polymerizable material comprising at least one gassing device for introduction of a gas into the polymerizable material and a heating device for heating the polymerizable material provided with gas. The invention also relates to a process for heating a polymerizable material, a device for production of high purity (meth)acrylic acid, and a process for production of high purity (meth)acrylic acid.

By (meth)acrylic acid is presently understood both methacrylic acid and acrylic acid, whereby acrylic acid is particularly preferred.

It is known that polymerizable materials such as (meth)acrylic acid and (meth)acrylic acid ester may easily be caused to polymerize by heat and/or the action of light and/or peroxides. Since, however, in production, processing and/or storage polymerization must be minimized or prevented for technical safety and economic reasons, there is a constant requirement for new, simple, and effective methods and installations for reduction of uncontrolled polymerization. It is, further, also known that the polymerization of (meth)acrylic acid and (meth)acrylic acid esters may be suppressed by use of polymerization inhibitors such as, for example, hydroquinones or hydroquinone derivatives. The presence of oxygen also inhibits polymerization.

Generally, (meth)acrylic acid produced by catalytic gas phase oxidation of unsaturated carbohydrates such as, for example, propylene or isobutylene. First a gaseous reaction mixture comprising (meth)acrolein is obtained, which is oxidized in a second oxidation step to obtain a (meth)acrylic acid-comprising product gas mixture. This product gas mixture is then condensed or absorbed in a suitable solvent, whereby a (meth)acrylic acid-comprising liquid phase is obtained. This liquid phase, which, besides the (meth)acrylic acid and optionally the absorption agent, still comprises numerous side products, which are formed in the gas phase oxidation of the unsaturated carbohydrates as starting compounds, is then purified by means of further purification processes, in particular by means of distillation. Optionally, the (meth)acrylic acid is also converted with suitable alcohols to the corresponding (meth)acrylic acid esters.

In the distillative purification of (meth)acrylic acid-comprising liquids, or in the esterification, these liquids must, however, be heated to a temperature necessary for a sufficient separation performance or respectively for an esterification, whereby this heating preferably occurs in suitable pipe bundle heat exchangers. In order to prevent a polymerization of the (meth)acrylic acid during the thermally stressing distillation or esterification respectively, it is also common to add polymerization inhibitors.

The currently known systems for processing of a polymerizable material still may not provide sufficient reliability with respect to polymerization occurring, in the region of the pipe bundle heat exchanger.

It is an object of the present invention to at least partially alleviate the technical problems described with reference to the prior art. In particular, an arrangement should be proposed, in which a treatment of the polymerizable material is provided with particularly little polymerization. The arrangement should, additionally be distinguished by a simple construction and low maintenance requirements. Lastly, particularly suitable processes and uses of this arrangement as well as products described therewith should further be described.

### SUMMARY

These objects are solved by an arrangement for treatment of a polymerizable material with the features of arrangement for treatment of a polymerizable material comprising at least one gassing device to feed a gas into the polymerizable material, and a heating device to heat the polymerizable material provided with gas, characterized in that the heating device is designed so that the polymerizable material flows through the heating device via an inlet against gravity and means for even distribution of the gas over the inlet are provided.

Another embodiment includes a process for heating a polymerizable material wherein the process includes heating of a polymerizable material, wherein the polymerizable material in an arrangement 1) with the features of arrangement for treatment of a polymerizable material comprising at least one gassing device to feed a gas into the polymerizable material, and a heating device to heat the polymerizable material provided with gas, characterized in that the heating device is designed so that the polymerizable material flows through the heating device via an inlet against gravity and means for even distribution of the gas over the inlet are provided is first enriched with a gas comprising oxygen and then heated.

### FIGURES

The foregoing and other features, aspects, and advantages of the present invention will become better understood with regard to the following description, appended claims, and accompanying drawing where:

FIG. 1 shows schematically, a first embodiment of an arrangement according to the invention;

FIG. 2 shows schematically, a detail of a further embodiment of the arrangement;

FIG. 3 shows an embodiment of a flow influencer;

FIG. 4 shows a further embodiment of the arrangement in detail;

FIG. 5 shows a schematic representation of an embodiment of a flow conditioner;

FIG. 6 shows schematically, a part of the (meth)acrylic acid production; and

FIG. 7 shows schematically, a flow profile through a flow conditioner.

### DETAILED DESCRIPTION

The arrangement according to an embodiment of the invention for treatment of a polymerizable material comprises at least one gassing device to feed a gas into the polymerizable material and a heating device to heat the polymerizable material provided with gas. The heating device may be designed so that the polymerizable material flows through the heating device via an inlet against gravity and means for even distribution of the gas over the inlet are provided.

The polymerizable material may be present substantially as fluid, whereby the gas may be introduced into this fluid by means of at least one gassing device. Such a gassing device may comprise respectively a single gassing position. It is also possible that more gassing positions may be achieved by means of an individual gassing device. In the latter case, it may be possible that each gassing position may be operated individually, in groups, or all together.

The heating device may be designed with heat exchanger surfaces that may be brought into contact with the polymerizable material. The heating device may be heated in any known way; preferred, however, is heating with water vapor. The heating device comprises separated flow paths for the water vapor and the polymerizable material. The heat exchange media (polymerizable material, water vapor) may be conducted to each other in any direction, in particular also in counter-current flow.

By inlet is meant the entry opening of the heating device. The inlet may be designed with a round cross-section, which has a diameter within the range from about 500 to about 2500 mm. The inlet may be arranged horizontally.

The arrangement may be designed so that the polymerizable material may flow against gravity or the polymerizable material may flow substantially parallel against gravity. This is advantageous if the flow paths for the polymerizable material through the heating device likewise run parallel to gravity. In this way, regions within the flow paths in which an increased accumulation of the supplied gas occurs are avoided. The even distribution of the gas in the polymerizable material remains over the total duration of the flowing through of the heating device. In this way, polymerization in the heating device is considerably reduced and partially even durably prevented.

The means for even distribution of the gas over the inlet ensure that each part-flow of the polymerizable material during flowing through the heating device may be provided with the same gas content, or with a gas which inhibits polymerization, and thus the same, low tendency to polymerize is present in all flow paths of the polymerizable material. The means may, e.g., be achieved with particularly suitable embodiments of the gassing device and/or of the inlet and/or separate components in the installation. Embodiments are described in the following.

The vertical construction of the arrangement, as well as the uniform supply or distribution respectively of the gas in the polymerizable material, may reduce the tendency to polymerize, so that the heating device works over a long time period with a high degree of effectiveness. In this way, costs and maintenance requirements may be observably reduced.

According to a further embodiment of the arrangement, it is proposed that the heating device comprises at least one vertical pipe bundle heat exchanger. With a pipe bundle heat exchanger, the polymerizable material is conducted through a plurality of pipes, around which a hot medium (e.g., water vapor) flows. Such a pipe bundle heat exchanger is preferably designed with a height within the range from about 4 to about 6 m. The horizontal extension lies, for example, within the range from about 1 to about 2.50 m. Such a pipe bundle heat exchanger may be placed in a container similar to a column. Besides the pipe bundle heat exchanger, lamellar bundle heat exchangers, which comprise narrow channels which are produced from sheet packets by point or seam welding, may also be used as heating device. With respect to the exact construction of pipe bundle or lamellar bundle heat exchangers respectively, reference is made to “Basic Operations of Chemical Process Technology” (“Grundoperationen Chemischer Verfahrenstechnik”), Wilhelm R. A. Vauck and Hermann A. Müller, Wiley VCH-Verlag, 11th revised and extended edition, 2000, pages 502-506. The disclosure of this textbook concerning and limited to the construction of pipe bundle and lamellar heat exchanges used in connection to this application is hereby introduced as reference and forms part of the disclosure of the present invention.

It is further proposed that the at least one gassing device is arranged at a distance to the inlet of the heating device which lies within a range from about 300 to about 1000 mm, or in a range from about 300 to about 500 mm. In this way, a particularly compact arrangement is created. In interplay with the means for even distribution of the gas, a very good distribution of the gas may be achieved despite such a small distance. The gassing device then leads, e.g. not into a pipe but into a type of mixing chamber in the transition region from the pipe to the heating device. Particularly small distances may be maintained if the gassing device is designed correspondingly, i.e., for example, already enables itself a distributed supply of the gas via more than one gassing position.

According to a further embodiment of the arrangement, the gassing device comprises at least one nozzle, which has an opening angle of at least 30°. Nozzles with an opening angle within the range from 40° to 50° may be used. In this way, the gas is advantageously piped with an over pressure of at least 2 bar, or within the range of 3 bar, into the fluid polymerizable material. With respect to the positioning of the at least one nozzle, it should be ensured that an even gassing of the inlet of the heating device is achieved. Thus, for example, an individual nozzle should be positioned as centrally to the inlet as possible, while several nozzles should be arranged evenly distributed either over the cross-section of the inlet and/or at the circumference. The nozzles may be arranged so that the flowing in of the gas occurs counter to the flow direction of the polymerizable material.

It is further advantageous that at least one flow influencer is provided between the gassing device and the inlet. The flow influencer has, in particular, the function of distributing or diverting the fluid polymerizable material. Possible embodiments of a flow influencer may include, for example but not limited to a sieve, a grating, or a hole plate. With respect to a hole plate, it is preferred that this has a free flow cross-section within the range up to about 30%. As hole diameter, a range from about 20 to about 30 mm is present. The flow influencer may also optionally at least partially serve as deflector for the gas and/or the polymerizable material, so that a turbulence or expansion of part-flows takes place. An embodiment of the flow influencer in which the total flow of the polymerizable material flows through it is preferred.

According to a further embodiment of the arrangement, before the gassing device, seen in the flow direction of the polymerizable material, at least one flow conditioner is provided for generation of a cross-flow. Such a flow conditioner may have an effect primarily on the flow of the polymerizable material. It effects a turbulence, rotation, or the like. In this way, it may be achieved that the polymerizable material in the region of the expansion before the inlet of the heating device is distributed evenly and quickly. It is also further possible that a specific flowing past of the polymerizable material at the gassing device takes place. It is further advantageous that the flow conditioner undertakes a type of flow guidance over a particular length in the flow direction. This length is advantageously not smaller than about 300 mm and may be larger than about 500 mm.

In this context, it is advantageous that the at least one flow conditioner comprises a plurality of deflectors for generation of a rotation of at least one part of the polymerizable material. In this way, it is achieved that with a frontal flowing of the polymerizable material towards the inlet of the heating device, a diversion in a cross-direction now takes place by means of the flow conditioner.

Furthermore, a process for heating of a polymerizable material is proposed, wherein the polymerizable material in an arrangement according to the invention is first enriched with a gas comprising oxygen and then heated.

The polymerizable material is preferably an ethylenically unsaturated compound selected from the group consisting of acrylic acid, methacrylic acid, acrylic acid esters, and methacrylic acid esters, whereby as methacrylic acid ester, methacrylic acid methyl ester and methacrylic acid ethyl ester and as acrylic acid ester, acrylic acid methyl ester, and acrylic acid ethyl ester are examples.

It is furthermore preferred according to the invention that the polymerizable material, into which the gas is introduced, comprises further side-products besides the above-mentioned ethylenically unsaturated compounds.

According to a particular embodiment of the process according to the invention, the polymerizable material is based upon
(α1) from about 50 to about 99.5 wt. %, or from about 60 to about 99 wt. % (meth)acrylic acid,
(α2) from 0.0001 to about 20 wt. %, or from about 5 to about 15 wt. % of a separating agent,
(α3) from 0.0001 to about 5 wt. %, or from 0.001 to about 1 wt. % water,
(α4) from 0.0001 to about 10 wt. %, or from about 0.5 to about 5 wt. % dimeric (meth)acrylic acid, and
(α5) from 0.1 to about 15 wt. %, or from 0.5 to about 10 wt. % further side-products,
   whereby the sum of the components (α1) to (α5) is 100 wt. %.

In the case of the treatment of acrylic acid as polymerizable material, the separating agent is preferably organic solvents with which water forms an azeotrope. An example of a separating agent is toluene. The side-products, in the case of the treatment of acrylic acid, may be compounds selected from the group comprising protoanemonin, propionic acid, maleic acid, maleic acid anhydride, aldehydes such as furfural, benzaldehyde or propionaldehyde and polymerization inhibitors such as hydroquinone or phenothiazine.

According to another particular embodiment of the process according to the invention, the polymerizable material may be based upon
(β1) from 0 to about 1 wt %, or from 0.0001 to 0.1 wt. % (meth)acrylic acid,
(β2) from about 50 to about 99 wt. %, or from about 60 to about 98 wt. % (meth)acrylic acid esters,
(β3) from 0.0001 to about 1 wt. %, or from 0.01 to about 0.5 wt. % water,
(β4) from 0 to about 2 wt. %, or from 0.0001 to about 1 wt. % of an alcohol, and
(β5) from about 0.5 to about 10 wt. %, or from about 1 to about 5 wt. % further side-products,
   whereby the sum of the components (β1) to (β5) is 100 wt. %

The alcohols include, but are not limited to primary alcohols selected from the group comprising methanol, ethanol, 1-propanol, 2-propanol, n-butanol, iso-butanol or tert-butanol, and the esters that include, but are not limited to methyl ester, ethyl ester, propyl ester or butyl ester. The side-products are, besides polymerization inhibitors, for example, compounds selected from di-n-butyl ether, n-butyl acetate, and butyl butyrate.

As gas that is introduced into the polymerizable material, oxygen or oxygen-comprising gas mixtures such as, for example, air may be used.

According to a another embodiment of the process according to the invention for heating of a polymerizable material, the polymerizable material may be the composition that is obtained as bottom product according to the following process:
A) catalytic gas phase oxidation of propylene to form an acrolein-comprising first product gas mixture;
B) catalytic gas phase oxidation of the acrolein to form an acrylic acid-comprising second product gas mixture;
C) condensation of the second product gas mixture in a quench tower to obtain an aqueous acrylic acid solution; and
D) azeotropic distillation of the aqueous acrylic acid solution in the presence of toluene as entrainer to obtain a bottom product and head products.

According to a another embodiment of the process according to the invention, the polymerizable material enriched with oxygen may be heated to a temperature to at least about 85° C., or to at least 90° C. Generally, the polymerizable material, if it is a polymerizable material processed again from a distillation column, flows in with a temperature of from about 70 to about 80° C. According to the process proposed here, a temperature increase to up to about 91° C. may be achieved, which is considerable taking into account the short residence within the heating device. The flow speed of the polymerizable material is, for example, from about 2 to about 5 m/s. This makes a particularly good heat transfer in the heating device necessary, which may be ensured with the above-mentioned measures, since polymerization no longer substantially hinders the heat transfer.

The polymerizable material may be supplied with an average volume flow of from about 500 to about 3000 m³/h. This has considerable economic consequences, since, for example, the production of acrylic acid may be carried out very quickly and on a large scale.

For the heating device, which is operated in cooperation with a distillation device, it is suggested with respect to the above-mentioned volume flows that the oxygen-comprising gas is supplied with an average mass flow of from about 60 to about 180 kg/h. This amount of a polymerization-inhibiting gas is, on the one hand, sufficient to avoid polymerization in the heating device and, on the other hand, small, since, because of the even distribution, an over-provision is not necessary.

The present invention also relates to a device for production of high purity (meth)acrylic acid, comprising as device components connected with each other in fluid-conveying fashion
(a) a synthesis reactor,
(b) an absorption or condensation device, and
(c) a purification device,
   wherein the purification device (c) comprises at least one arrangement according to the invention for treatment of a polymerizable material.

As reactor (a), all reactors known to the skilled person may be used which are generally used in the production of (meth)acrylic acid. Preferred reactors are pipe bundle reactors with a catalyst packed bed and reactors which comprise thermoplates coated with catalyst or thermo-plates with a catalyst-packed bed fill. The reactors also comprise, in addition to the reaction space associated with the catalyst, a heat transport space through which a cooling medium flows. Preferred reactors according to the invention are described, for example, in EP-A-0 700 893 and in DE-A-101 08 380.

The absorption or condensation device (b) is preferably a so-called “quench tower”, as described in EP-A-1 319 648. In such a quench tower, the (meth)acrylic acid obtained in the second oxidation step (in which (meth)acrolein is oxidized to (meth)acrylic acid) and the reaction water arising during the oxidation are subjected to a total condensation to form an aqueous (meth)acrylic acid solution. In principle, condensation devices may be condensers with components which are effective for separation, in particular with packings, filling materials and/or floors, for example bubble trays, sieve floors, valve floors and/or dual-flow floors. In this way, the condensable components of the gaseous product mixture may be fractionally condensed out by cooling. Since the gas mixture comprises a high-boiling, mid-boiling and low-boiling fraction as well as non-condensable components as a result of the impurities and dilution gases, in the column, at the corresponding positions, one or more side outlets may be provided. Contrary to a general condensation, a condensation in a column thus already enables a separation into the individual components. Suitable columns comprise at least one cooling device, whereby all common heat transferers or heat exchangers, with which the heat formed during the condensation is removed indirectly (externally) are suitable. Pipe bundle heat exchangers, plate heat exchangers and air coolers are preferred. Suitable cooling media are air for the corresponding air cooler and cooling fluids, in particular water, for other cooling devices. If only one cooling device is provided, this is fitted at the head of the column in which the low-boiling fraction is condensed out. Since the (meth)acrylic acid-comprising gas mixture comprises several fractions, it is practical to fit a plurality of cooling devices in different sections of the column, e.g. a cooling device in the lower section of the column for condensing out the high-boiling fraction and a cooling device at the head of the column for condensing out the low-boiling fraction. In the case of the production of acrylic acid, this is removed via one or more side outlets in a fraction in the middle part of the column.

Besides the condensation of the gaseous reaction components, these may also be intimately brought into contact with an absorption fluid in an absorption column provided with components which are effective for separation and absorbed in this way.

The aqueous acrylic acid solution obtained in the quench tower, the acrylic acid removed in the condensation column as side stream or the acrylic acid solution obtained after absorption in a suitable solvent may then be further purified in a suitable purification device (c). This purification device (c) comprises at least one arrangement according to the invention for treatment of a polymerizable material. According to a particular embodiment of the device according to the invention for (meth)acrylic acid, the purification device (c) comprises a distillation device, which is connected with an arrangement according to the invention in such a way that at least a part of the bottom product of the distillation device is removed from the distillation device by means of a conveying element, preferably by means of a pump, treated in an arrangement according to the invention, i.e. brought into contact with a gas and heated, and then conducted back into the distillation device.

According to a particular embodiment of the device according to the invention, the condensation device (b) is a quench tower and the purification device (c) is a distillation column, in which, in the presence of toluene as entrainer, the aqueous acrylic acid solution obtained in the quench tower is azeotropically distilled.

The invention also relates to a process for the production of high-purity (meth)acrylic acid comprising the process steps:
(A) production of a (meth)acrylic acid-comprising product gas mixture in a reactor, and
(B) purification of the product gas mixture to obtain a (meth)acrylic acid with a purity of at least 99.5 wt. %, particularly preferably at least 99.8 wt. % and yet more preferably at least 99.9 wt. % in a purification device comprising at least one arrangement according to the invention.

According to process step (A), first, in a reactor according to the invention, a (meth)acrylic acid-comprising product gas mixture is produced. Preferably, two reactors connected in a row are used, whereby in the first reactor, the oxidation of propylene or isobutylene respectively or of other suitable starting compounds to (meth)acrolein occurs and in the second reactor the conversion of (meth)acrolein to (meth)acrylic acid occurs. It is, however, also conceivable to carry out the oxidation to (meth)acrylic acid in one step in a reactor according to the invention.

In process step (B), the purification of the (meth)acrylic acid-comprising product gas mixture occurs. Here, at least one product stream comprising (meth)acrylic acid and arising during the purification of the (meth)acrylic acid is treated in a device according to the invention. The purification may comprise at least one distillation step.

The present invention also relates to the high purity (meth)acrylic acid obtainable by the above-described process.

In addition, the invention relates to a process for production of a (meth)acrylic acid-comprising polymer, whereby a high purity (meth)acrylic acid obtainable by the above-described process is polymerized. The polymerization may occur as solution polymerization, whereby the reaction guide in a troughed belt conveyor is an example. In this case the aqueous phase may be used directly or the water-poor phase may be correspondingly diluted. In general, the polymerization occurs in a medium with a water content of from about 20 to about 80 vol %, based on the medium.

Furthermore, the invention relates to a polymer obtainable according to the process described in the above paragraph.

The polymer is preferably an absorbing polymer with a maximum absorption of 0.9 wt. % aqueous NaCl solution according to ERT 440.1-99 within a range from about 10 to about 1000 ml/g, or from about 15 to about 500 ml/g, or from about 20 to 300 ml/g. More details concerning absorbing polymers and their production may be found in “Modern Superabsorbent Polymer Technology”, F. L. Buchholz, A. T. Graham, Wiley-VCH, 1998.

The high purity (meth)acrylic acid according to the invention or the polymer according to the invention may be used in or for production of fibers, formed bodies, films, foams, superabsorbing polymers, or hygiene articles.

The invention and the technical field may be more closely illustrated in the following by means of the figures, without restricting the invention to these. It should be mentioned that the size relationships illustrated in the figures do not represent the actual size relationships, unless this is explicitly mentioned in the figure description. The figures show:

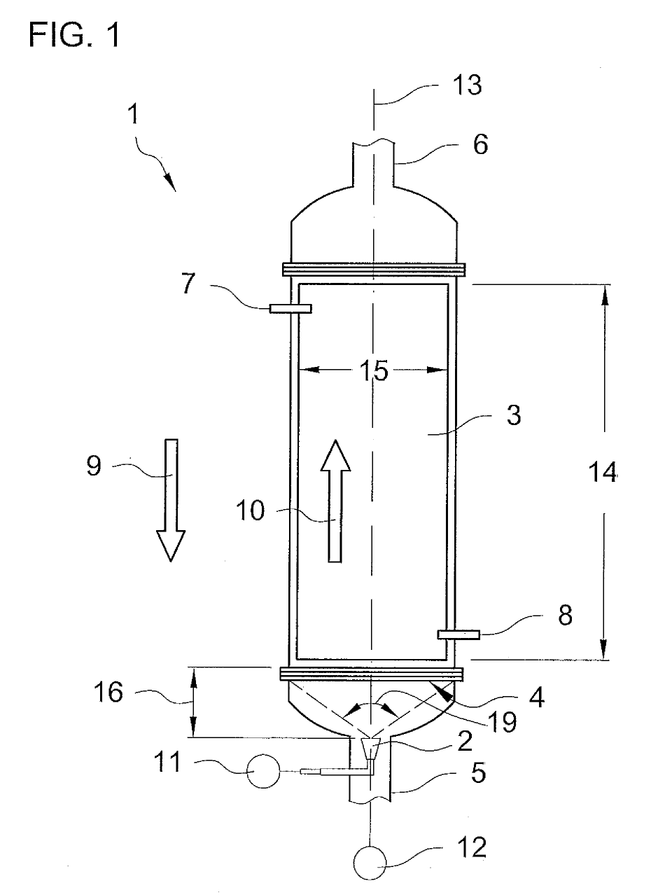

FIG. 1 shows an arrangement 1 for treatment of a polymerizable material 12 with a gassing device 2 for introduction of a gas 11 into the polymerizable material 12 and a heating device 3 for heating the polymerizable material 12 provided with gas 11. The heating device 3 is designed so that the polymerizable material 12 flows through the heating device 3 via an inlet 4 against gravity 9.

The polymerizable material 12 flows past as a fluid through the reactant entry 5 at the gassing device 2. The gassing device 2 is here designed with a nozzle system arranged centrally to axis 13. The gassing device 2 distributes the gas 11 (in particular a polymerization inhibitor comprising oxygen) with an opening angle 19 in the fluid, polymerizable material 12. The gassing device 2 is positioned with a distance 16 to the inlet 4 of the heating device 3, such that the opening angle 19 reaches the entire cross-section of the inlet 4. Suitable means for even distribution of the gas in the polymerizable material are therewith provided.

The stream of the polymerizable material 12 enriched with the gas 11 now enters into heating device 3 and flows through with flow direction 10, which runs substantially parallel and opposite to gravity 9. Inside the heating device 3, the polymerizable material 12 is conducted in separated flow paths to product outlet 6. The heating occurs via a vapor-like medium, which is supplied via a vapor entry 7 and removed via a condensate exit 8. In this way, a heating distance arises, which substantially corresponds to the height 14 of the heating device 3. In general, such a heating device 3, in particular when it is designed as a pipe bundle heat exchanger, is designed with an extension 15 within the range from 500 mm to 2500 mm.

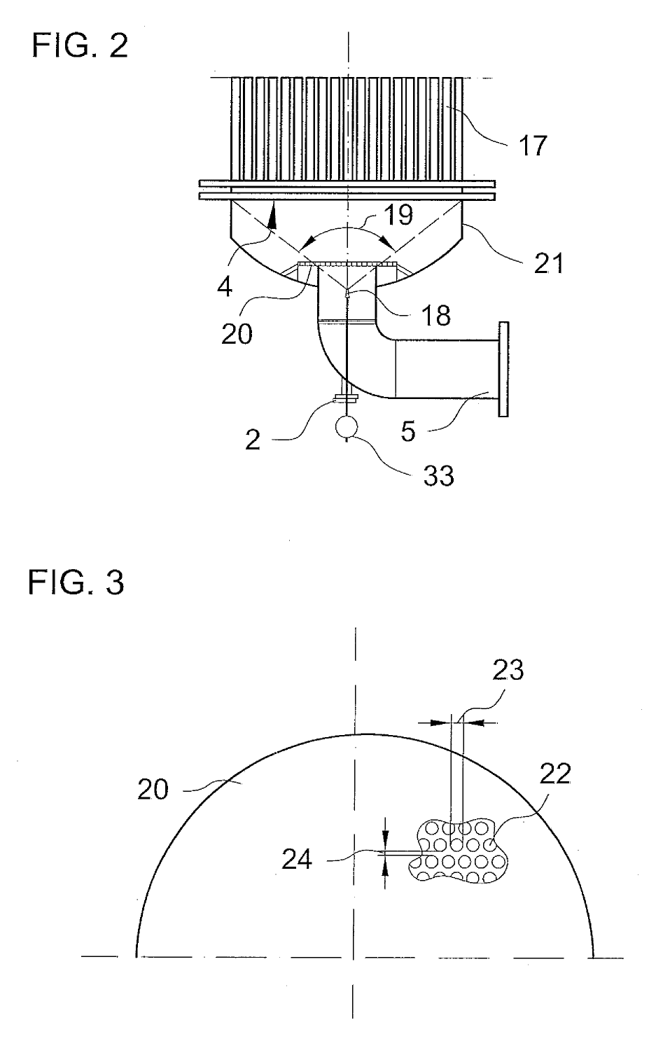

FIG. 2 shows a detail of a further embodiment of an arrangement, with which the reactant entry 5 is bent and in the curve a supply for the gassing device 2 is provided. The gassing device 2 in turn distributes the gas 11 in the polymerizable material 12, whereby the opening angle 19 is of such dimensions that the inlet 4 is closed. To this end, the gassing device 2 is operated with a conveyor 33 for the gas 11, which ensures an over-pressure within the reactant entry 5 or the mixing chamber 21 respectively of at least 2 bar. The gassing device 2 comprises an individual nozzle 18, which is preferably designed as a conical jet nozzle and positioned centrally. p Downstream of nozzle 18, the polymerizable material 12 and the gas 11 first flow through a flow influencer 20, which is designed as hole plate. The flow influencer 20 effects a turbulence of part-flows after exit from the flow influencer 20 so that an even distribution of the gas-material mixture takes place. Thus substantially the same concentration of the gas 11 in the pipes of the pipe bundle heat exchanger 17 is assured.

A partial view of such a flow influence 20 is illustrated in FIG. 3. The flow influencer comprises a plurality of openings 22, which, for example, have an extension 23 of about 25 mm. The openings 22 should, for example, be positioned at such a distance 24 to each other that a uniform distribution of the openings 22 is given, whereby the sum of the openings 22 makes up about 30% of the total surface of the flow influencer 20.

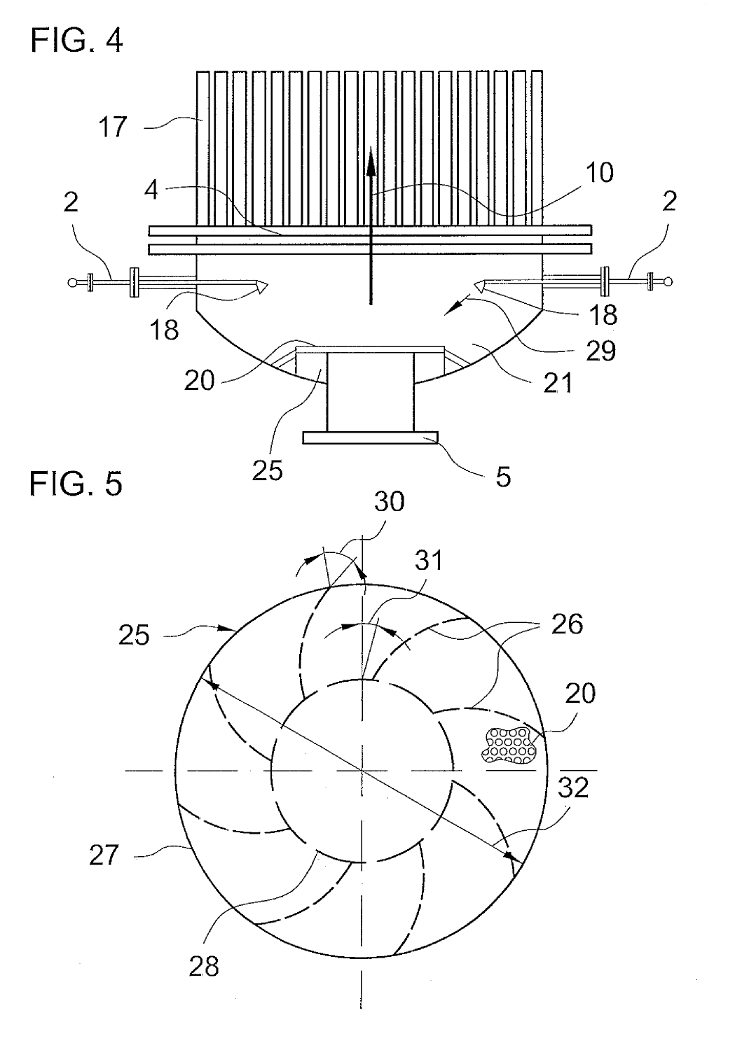

FIG. 4 depicts a further embodiment of an arrangement 1 with a plurality of gassing devices 2. Here, the polymerizable material 12 first flows over the reactant entry 5 into the mixing chamber 21 before the inlet 4 of the pipe bundle heat exchanger 17. A flow influencer 25 is provided in the transition from the reactant entry 5 to the mixing chamber 21. This has the effect that at least parts of the polymerizable material 12 flowing in are diverted to the gassing device 2. In this way, the polymerizable material 12 flows counter to the gassing direction 29 and an even distribution may be achieved.

FIG. 5 shows an embodiment of such a flow conditioner 25. This is constructed with an outer cylinder 27 and an inner cylinder 28, between which a plurality of deflectors 26 are provided. The deflectors 26 are fixed with an outer angle 30 at the outer cylinder 27 and an inner angle 31 at the inner cylinder 28. In order to divert the flow in the radial direction, the flow conditioner 25 comprises a flow influencer 20 as a type of “lid”. The impact pressure generated therewith has the effect that parts of the stream move along the deflectors 26 and in this way are caused to rotate. Generally, such a flow conditioner 25 has a diameter 32 which is sufficiently large to cover the reactant entry 5.

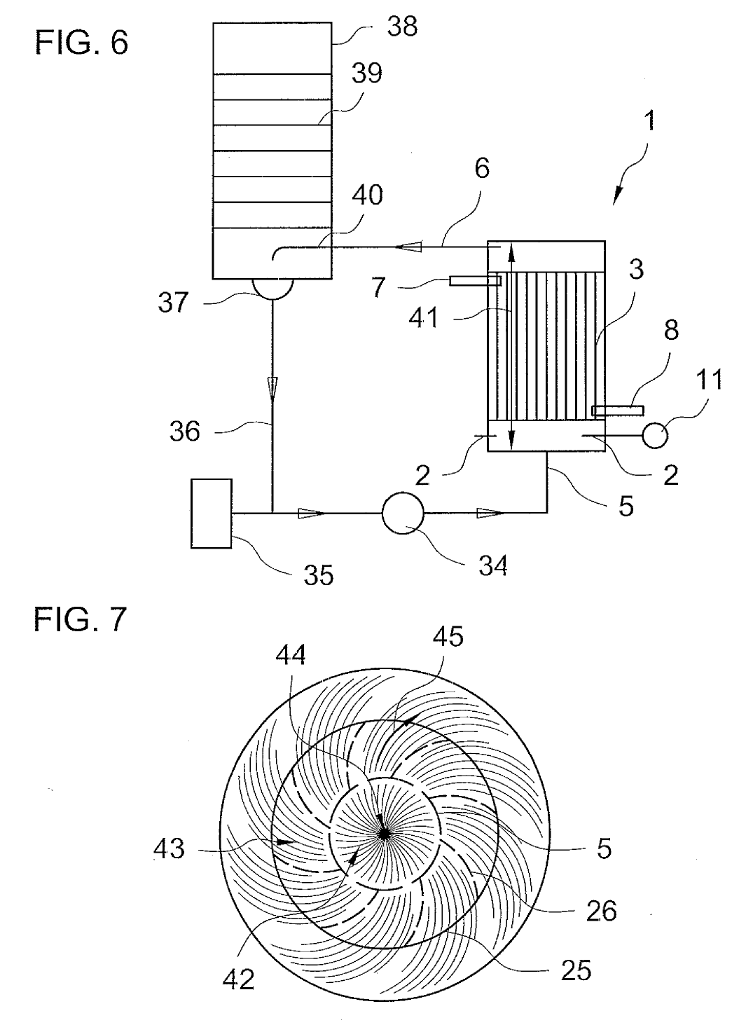

FIG. 6 illustrates the material transport through the arrangement 1 and a subsequently arranged distillation column 38, as is common in the production of acrylic acid. The polymerizable material 12 is supplied by means of a pump 34 and with a temperature within the range of about 80° C. via reactant entry 5 of the gassing device 2 of the gases 11 (inhibitor). The gas-material mixture then flows through a heating device 3. The total arrangement 1 has a dimension 41 of about 10 m. With an arrangement 1 of this type, particularly high volume flows of the polymerizable material 12 are heated in a very short time to temperatures at the product exit 6 within the range of 90° C. The residence of the polymerizable material lies in ranges below 10 seconds, depending on the dimension 41 of the arrangement 1. The heated, still fluid gas-material mixture then flows to the distillation column 38 and is introduced via an inlet 40. During this, the fluid pressure reduces and it rises upwards as vapor through the individual separating floors 39 of the distillation column 38. The condensate collects in a collecting reservoir 37 of the distillation column 38 and is fed back to the pump 34 via an outflow 36. This circuit may also be supplemented with a reservoir 35, which makes available additional polymerizable material 12 if necessary.

FIG. 7 should show the flow relationships in a flow conditioner 25. The flow conditioner 25 is drawn with its deflectors 26. In the center, the reactant entry 5 is marked with a dashed line. The amounts of the polymerizable material 12 flowing in the central area is at least partially diverted by means of the flow influencer 20 (not shown here) applied above and conducted to the deflectors 26. Thus, in the representation in FIG. 7, a calm region 44 may be made out in proximity to the centre, while at the edge region of the reactant entry 5 the normal flow 42 is formed. Upon flowing through the flow conditioner 25 or the deflectors 26 respectively, a flow acceleration 43 occurs, which is symbolized by dark regions. At the same time, a rotation 45 of the polymerizable material 12 occurs. This results in a particularly even movement of the polymerizable material 12 in the mixing chamber, so that here the polymerization inhibitor may be applied particularly well distributed.

### LIST OF REFERENCE NUMERALS

1 arrangement
2 gassing device
3 heating device
4 inlet
5 reactant entry
6 product exit
7 vapor entry
8 condensate exit
9 gravity
10 flow direction
11 gas
12 material
13 axis
14 height
15 extension
16 distance
17 pipe bundle heat exchanger
18 nozzle
19 opening angle
20 flow influencer
21 mixing chamber
22 opening
23 extension
24 distance
25 flow conditioner
26 deflector
27 outer cylinder
28 inner cylinder
29 gassing direction
30 outer angle
31 inner angle
32 diameter
33 conveyor
34 pump
35 reservoir
36 outflow
37 collecting reservoir
38 distillation column
39 separating floor
40 supply
41 dimension
42 normal flow
43 flow acceleration
44 calm region
45 rotation

1. Arrangement (1) for treatment of a polymerizable material (12) comprising at least one gassing device (2) to feed a gas (11) into the polymerizable material (12) and a heating device (3) to heat the polymerizable material (12) provided with gas (11), characterized in that the heating device (3) is designed so that the polymerizable material (12) flows through the heating device (3) via an inlet (4) against gravity (9) and means for even distribution of the gas (11) over the inlet (4) are provided.

2. Arrangement (1) according to claim 1, characterized in that the heating device (3) comprises at least one vertical pipe bundle heat exchanger (17).

3. Arrangement (1) according to claim 1 or 2, characterized in that the at least one gassing device (2) is arranged at a distance (16) to the inlet (4) of the heating device (3) which lies within a range from 300 to 1000 mm.

4. Arrangement (1) according to any one of the preceding claims, characterized in that the gassing device (2) comprises at least one nozzle (18) which has an opening angle (19) of at least 30°.

5. Arrangement (1) according to any one of the preceding claims, characterized in that at least one flow influencer (20) is provided between the gassing device (2) and the inlet (4).

6. Arrangement (1) according to any one of the preceding claims, characterized in that before the gassing device (2), seen in the flow direction (10) of the polymerizable material (12), at least one flow conditioner (25) is provided for generation of a cross-flow.

7. Arrangement (1) according to claim 6, characterized in that the at least one flow conditioner (25) comprises a plurality of deflectors (26) for generation of a rotation of at least a part of the polymerizable material (12).

8. Process for heating of a polymerizable material (12), wherein the polymerizable material (12) in an arrangement 1) defined in any one of claims 1 to 7 is first enriched with a gas (11) comprising oxygen and then heated.

9. Process according to claim 8, wherein the polymerizable material (12) enriched with oxygen is heated to a temperature of at least 80° C.

10. Process according to claim 8 or claim 9, wherein the polymerizable material (12) is (meth)acrylic acid.

11. Process according to any one of claims 8 to 10, characterized in that the polymerizable material (12) is supplied with an average volume flow within the range from 500 to 3000 m³/h.

12. Process according to any one of claims 8 to 11, characterized in that the oxygen-comprising gas (11) is supplied with an average mass flow of 60 to 180 kg/h.

13. A device for production of high purity (meth)acrylic acid, comprising as device components connected with each other in fluid-conveying fashion
(a) a reactor
(b) an absorption or condensation device, and
(c) a purification device
wherein the purification device (c) comprises at least one arrangement (1) according to any one of the claims 1 to 7.

14. A process for production of high purity (meth)acrylic acid comprising the process steps:
(A) production of a (meth)acrylic acid-comprising product mixture in a reactor,
(B) purification of the product gas mixture to obtain a (meth)acrylic acid with a purity of at least 99.5 wt. % in a purification device comprising at least one arrangement according to any one of claims 1 to 7.
